SEQUENCE LISTING (16 sequences)

SEQ ID NO: 1 - protein, 14 aa, Artificial Sequence
  LMWP
VSRRRRRRGGRRRR

SEQ ID NO: 2 - protein, 13 aa, Artificial Sequence
  Tat48-60
GRKKRRQRRRPPQ

SEQ ID NO: 3 - protein, 31 aa, Artificial Sequence
  Tat48-60-P10
GRKKRRQRRRPPQRQTSMTDFYHSKRRLIFS

SEQ ID NO: 4 - protein, 12 aa, Artificial Sequence
  CAI
ITFEDLLDYYGP

SEQ ID NO: 5 - protein, 9 aa, Artificial Sequence
  HIV-TAT
RKKRRQRRR

SEQ ID NO: 6 - protein, 18 aa, Artificial Sequence
  MAP
KLALKLALKALKAALKLA

SEQ ID NO: 7 - protein, 27 aa, Artificial Sequence
  MPG
GALFLAFLAAALSCMGLWSQPKKKRKV

SEQ ID NO: 8 - protein, 22 aa, Artificial Sequence
  M918
MVTVLFRRLRIRRACGPPRVRV

SEQ ID NO: 9 - protein, 22 aa, Artificial Sequence
  R6Pen
RRRRRRRQIKIWFQNRRMKWKK

SEQ ID NO: 10 - protein, 16 aa, Artificial Sequence
  Penetratin
RQIKIWFQNRRMKWKK

SEQ ID NO: 11 - protein, 21 aa, Artificial Sequence
  Pep-1-K
KKTWWKTWWTKWSQPKKKRKV

SEQ ID NO: 12 - protein, 22 aa, Artificial Sequence
  ARF1-22
MVRRFLVTLRIRRACGPPRVRV

SEQ ID NO: 13 - protein, 21 aa, Artificial Sequence
  Tp10
AGYLLGKINLKALAALAKKIL

SEQ ID NO: 14 - protein, 35 aa, Artificial Sequence
  POD
GGGARKKAAKAARKKAAKAARKKAAKAARKKAAKA

SEQ ID NO: 15 - RNA, 21 nt, Artificial Sequence
  Positive-sense strand
gauagagcaagacaagaaauu

SEQ ID NO: 16 - RNA, 21 nt, Artificial Sequence
  Antisense strand
uucuaucucguucuguucuuu

(19) United States
(12) Patent Application Publication
(10) Pub. No.: US 2022/0047713 A1
(43) Pub. Date: Feb. 17, 2022

(54) CELL-PENETRATING PEPTIDE-MULTIARM POL YETHYLENE GLYCOL-DRUG CONJUGATE HAVING TARGETING PROPERTY AND APPLICATION THEREOF

(71) Applicant: JENKEM TECHNOLOGY CO., LTD. (BEIJING), Beijing (CN)

(72) Inventors: Meina LIN, Beijing (CN); Xuan ZHAO, Beijing (CN); Zhengang ZHU, Beijing (CN)

(73) Assignee: JENKEM TECHNOLOGY CO., LTD. (BEIJING), Beijing (CN)

(21) Appl. No.: 17/044,866

(22) PCT Filed: Apr. 2, 2019
(86) PCT No.: PCT/CN2019/081057

§ 371 Date: Oct. 2, 2020

(30) Foreign Application Priority Data
Apr. 2, 2018 (CN) 201810282777.X

(51) Int. Cl. A61K 47/68; A61K 47/60; A61K 47/64; A61K 47/54

(52) U.S. Cl. CPC A61K 47/6807; A61K 47/60; A61K 47/6835; A61K 47/54; A61K 47/64

(57) ABSTRACT

The present invention provides a cell-penetrating-peptide multi-arm-PEG medicine conjugate having a general formula I, II, III, IV or V. As compared with linear-chain-type PEG-cell-penetrating-peptide conjugates, the multi-arm PEG has multiple end groups, and has introduction sites for multiple functional groups, which can connect to multiple different active groups. In addition, the cell-penetrating-peptide multi-arm-PEG medicine conjugate to enable the medicine to enter the pathogenetic cells in a targeting manner, to achieve precise treatment. The present invention further provides use of a cell-penetrating-peptide multi-arm-PEG medicine conjugate having targeting ability in preparation of a targeting medicine, especially use of a cell-penetrating-peptide multi-arm-PEG medicine conjugate having targeting ability in the treatment of eye age-related macular degeneration, asthma and pulmonary fibrosis.

### TECHNICAL FIELD

The present invention relates to the field of biological medicines, and particularly relates to a cell-penetrating-peptide multi-arm-polyethylene-glycol medicine conjugate, especially a cell-penetrating-peptide multi-arm-polyethylene-glycol medicine conjugate having a targeting ability and use of the cell-penetrating-peptide multi-arm-polyethylene-glycol medicine conjugate in biological medicine.

### BACKGROUND

Cell penetrating peptides (CPPs) are a type of polypeptides that, in a non-receptor-dependent manner and a non-typical endocytosis manner, directly pass through a cell membrane and enter the cell. They have lengths generally not exceeding 30 amino acids and are rich in basic amino acids, and the amino-acid sequences are generally positively charged. Currently, scientists have already applied them for gene therapy. To penetrate a cell membrane and enter the cell is the precondition that many effect targets play a role in biomacromolecules inside cells. However, the effect of biological barrier of biofilms prevents many polymer substances from entering cells, which restricts to a large extent the application of those substances in the field of treatment.

The non-patent literature “Progress in the Study of Cell penetrating Peptides in Medicine Delivery Systems” (Fan Bo, et. al, Acta Pharmaceutica Sinica, 2016(2): 264-271) describes that, currently, the CPPs that have already been found mainly include transcribed trans-activators (Tat), VP22, transportan, membrane-type amphiphilic peptides (MAP), signal transduction peptides and arginine-sequence-rich peptides. According to the features in terms of the amino-acid sequences, the hydrophobicities, the polarities and so on, CPPs can be generally classified into 3 types: cationic peptides, mainly including: R9, TAT, hLF, (RXR)4, NLSs and AMPs; amphipathic peptides, mainly including: MPG, penetratin, CADY, vascular endothelial-cadherin (pVEC), ARF (1-22) and BPrPr (1-28); and hydrophobic peptides, mainly including a signal sequence found in the integrin β3 (VTVLALGALAGVGVG) and a Kaposi fibroblast growth factor (AAVALLPAVLLALLAP).

CPPs have powerful potential in transportation, which enables them to become a good carrier of targeting medicines. So far, CPPs have effectively mediated various types of substances having bioactivity of different molecular weights and particle sizes to enter cells, such as small-molecule medicines, dyes, polypeptides, polypeptide nucleic acids, proteins, antibodies, plasmid DNAs, small interfering RNAs (siRNA), liposomes, bacteriophage particles, superparamagnetic particles, fluorescent stains, nanoparticles, viruses, quantum dots and magnetic-resonance-imaging contrast mediums.

CPPs can deliver multiple types of substances to enter cells. Because the physicochemical properties of the delivered substances are not the same, different linkage modes are required to link CPPs and the delivered substances, and generally the linkage modes influence significantly the ingestion amounts and the ingestion modes of CPPs. Commonly used linkage modes are covalent-bond linkage and static-electricity-effect linkage. Currently, many studies are on the delivery of nucleic-acid-type medicines mediated by CPPs. It can be seen from those studies that nucleic-acid-type medicines have strong electronegativities, and can have interattraction with electropositive CPPs to form a hairpin structure, which prevents the realizing of intracellular delivery without affecting the medicine activity. Coupling a polyethylene glycol (PEG) soft segment between the CPPs and the nucleic-acid-type medicines can solve that problem. The soft segment can separate the nucleic-acid-type medicines and the CPPs in the physical space, and in turn prevent the strongly electronegative nucleic-acid-type medicines and the electropositive CPPs from interattracting to form the hairpin structure, which prevents the aggregation and precipitation effect between the nucleic-acid-type medicines and the CPPs due to the static electricity effect, to enable the nucleic-acid-type medicines to, after entering cells, exploit their bioactivities inside the cytoplasms or cell nucleuses, to improve their bioavailabilities.

PEG is a polyether polymer compound that has extremely extensive uses, and can be applied in many fields such as medicine, hygiene, food and chemical engineering. PEG can be dissolved in water and many solvents. Furthermore, the polymer has an excellent biocompatibility, and, in vivo, can be dissolved in tissue fluid, and can be rapidly discharged from the body, without generating any toxic and side effects.

In the prior arts in which CPPs are linked to PEG as the medicine delivery system, all of the involved PEGs are of a straight-chain-type structure. For example, the patent literature CN 105727304 A introduces preparation and use of a nucleic-acid conjugate, wherein a particular structure of the nucleic-acid conjugate is that two terminals of a soft segment are separately covalently-linked to a cell penetrating peptide and a nucleic-acid-type medicine, wherein the soft segment is formed by any one of PEG, polyoxyethylene, polyoxypropylene, polyethylene and polyacrylamide that have a linear structure. As another example, the patent literatures EP 1797901 A1 and US 2013137644 A1 describe using a hydrophilic polymer as a linking group to link a CPP and a nucleic-acid-type medicine, to improve the delivery efficiency of a medicine inside a cell, wherein the hydrophilic polymer is preferably PEG, and is of a linear-chain-type structure. It can be seen that all of the existing reports utilize linear-chain-type PEGs as the linking group to link CPPs and treatment medicines, and do not make any improvement on the loaded dosage in the delivery systems formed by CPPs. However, linear-chain-type PEGs can link to one CPP and one medicine molecule at the two terminals of one molecule, and in each of the molecules, the linking rates of the CPPs or the medicines are low. In the present invention, the inventor has modified linear-chain PEGs into multi-arm PEG, which has multiple end groups, and has introduction sites for multiple functional groups, which can connect to multiple different active groups, which solves the problems of linear-chain-type PEGs on limited linkage sites, a small scope of application and a low medicine loading capacity.

In the practical application of cell penetrating peptides, the investigators have found another problem. Cell penetrating peptides have no selectivity to cells, and can carry medicines to enter all cells, without definite purpose. It frequently happens that pathogenetic cells and normal cells are killed together, whereby the best efficacy of the carried medicines is not exploited, and the body is harmed to a certain extent. Therefore, it is a problem to be urgently solved to invent a delivery system that can introduce medicines into pathogenetic cells in a targeting manner and can exploit the best effect of the medicines. The non-patent literature “Application of Cell penetrating Peptides in the Targeting Treatment of Tumors, Zhang Li, et. al, Cancer Research On Prevention and Treatment, 2015,42(10): 1043-1048” records that cell penetrating peptides are applied in the targeting treatment of tumors mainly in three aspects: firstly, using acid sensitive cell penetrating peptides to modify anti-tumor medicines to improve the targeting ability; secondly, using targeting cell penetrating peptides to modify anti-tumor medicines to improve the selectivity of the anti-tumor medicines; and thirdly, realizing targeted delivery by using cell penetrating peptides based on the specific receptors on the surface of tumor cells. For example, by modifying liposomes by using a dual-ligand formed by folic acid and the cell penetrating peptide PEP-1, anti-tumor medicines can be delivered into tumor cells by using such a delivery system, to efficiently and selectively kill the tumor cells, and reduce the adverse reactions of the medicines. However, the targeting group is merely linked to the cell penetrating peptide, and the linkage sites are limited.

In order to overcome the defects of the prior art, in the present invention, the inventor has designed a cell-penetrating-peptide multi-arm-polyethylene-glycol medicine conjugate that has targeting ability and can strongly enter cells. The medicine carrying rate is improved, and targeting groups can be linked to the cell penetrating peptide and/or the polyethylene glycol chain and/or the medicine molecule, whereby the medicine can enter the pathogenetic cells in a targeting manner, to exploit the best efficacy.

### SUMMARY

An object of the present invention is to provide a cell-penetrating-peptide multi-arm-PEG medicine conjugate. As compared with linear-chain-type PEG-cell-penetrating-peptide conjugates, the multi-arm PEG has multiple end groups, and has introduction sites for multiple functional groups, which can connect to multiple different active groups, which solves the problems of linear-chain-type PEGs on limited linkage sites, a small scope of application and a low medicine loading capacity.

Another object of the present invention is to provide a cell-penetrating-peptide multi-arm-PEG medicine conjugate having targeting ability. According to the particular demands of treatment, targeting groups can be connected to the cell penetrating peptide or the medicine molecule, to enable the medicine to enter the pathogenetic cells in a targeting manner, to achieve precise treatment.

Another object of the present invention is to provide a cell-penetrating-peptide multi-arm-PEG medicine conjugate having targeting ability. According to the particular demands of treatment, targeting groups can be connected to the chain ends of the multi-arm PEG, to enable the medicine to enter the pathogenetic cells in a targeting manner, to enable the medicine to exploit the best efficacy.

A still another object of the present invention is to provide use of the cell-penetrating-peptide multi-arm-PEG medicine conjugate having targeting ability, especially use of the cell-penetrating-peptide multi-arm-PEG medicine conjugate having targeting ability in the treatment of asthma and pulmonary fibrosis.

A cell-penetrating-peptide multi-arm-PEG medicine conjugate having a general formula

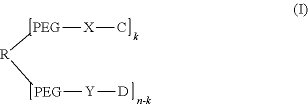

wherein R is a center molecule, and is selected from a polyhydroxy structure, a poly-amino structure or a poly-carboxyl structure;

preferably, R is selected from: a pentaerythritol or polypentaerythritol structure, a glycerol or polyglycerol structure, methyl glucoside and sucrose; and in a preferable embodiment of the present invention, R is selected from:

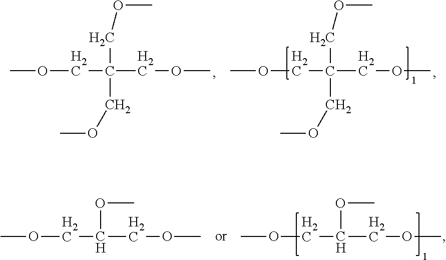

wherein l is an integer greater than or equal to 1 and smaller than or equal to 10; preferably, l is an integer greater than or equal to 1 and smaller than or equal to 10; more preferably, l is an integer greater than or equal to 1 and smaller than or equal to 7; and in a particular embodiment of the present disclosure, l is preferably 1, 2, 3, 4, 5 or 6.

The PEGs are the same or different —(CH₂CH₂O)ₘ—, and an average value of m is an integer of 3-250. Preferably, m is an integer of 68-250. More preferably, m is an integer of 68-227.

C is a CPP, and is selected from a transcribed trans-activator (Tat), VP22, transportan, a membrane-type amphiphilic peptide (MAP), a signal transduction peptide and an arginine-sequence-rich peptide;

preferably, C is selected from: LMWP, Tat48-60, Tat48-60-P10, CAI, HIV-TAT, MAP, MPGα, M918, R6Pen, penetratin, Pep-1-K, ARF1-22, Tp10, POD, polylysine formed by 3-100 lysine residues, and polyarginine formed by 4-9 arginine residues; and

more preferably, C is LMWP, or polyarginine formed by 8 arginines.

D is a medicine molecule, and the medicine molecule is selected from: a small-molecule medicine, a dye, a polypeptide, polypeptide nucleic acid, a protein, an antibody, a plasmid DNA, nucleic acid, liposome, bacteriophage particles, superparamagnetic particles, a fluorescent stain, nanoparticles, a virus, quantum dots and a magnetic-resonance-imaging contrast medium, especially siRNA, more especially Cytokine-siRNA;

preferably, D is selected from a small-molecule medicine, a polypeptide, an antibody and nucleic acid, wherein the nucleic acid includes a nucleotide monomer and an oligonucleotide; wherein the nucleotide monomer includes four deoxyribonucleotide monomers and four ribonucleotide monomers; and the oligonucleotide is a substituted oligonucleotide or an un-substituted oligonucleotide, wherein the substituted oligonucleotide is phosphorodiamidate morpholino oligomer, and the un-substituted oligonucleotide is selected from locked nucleic acid, siRNA, microRNA, an aptamer, peptide nucleic acid, decoy ODN, catalytic RNA and CpG dinucletide; and

more preferably, D is selected from a monoclonal antibody and siRNAs having a length of oligonucleotide of 19-23 bp.

In a preferable embodiment of the present invention, D is selected from Omalizumab, Nintedanib, Bevacizumab, Pembrolizumab, Trastuzumab, Nivolumab, VEGF-siRNA, IL-v-siRNA, Syk-siRNA and GATA-3-siRNA, wherein v is selected from 4, 5, 8 and 13.

X is a linking bond between the PEG and CPP, and the linking bond is formed by one or two or more of an amido bond, a disulfide bond, a hydrazone bond, an ester bond, a thioester bond, a mercapto-maleimide bond, -triazole-, a carbon-sulphur bond and an ether bond;

preferably, X is selected from one or a combination of two or more of —(CH₂)ⱼCONH(CH₂)ⱼ—, —(CH₂)ⱼ—S—S—(CH₂)ⱼ—, —(CH₂)ⱼNH—N═C(CH₂)ⱼ—, —(CH₂)ⱼCOO(CH₂)ⱼ—, —(CH₂)ⱼ—S—(CH₂)ⱼ—, -triazole- and a mercapto-maleimide bond; and

more preferably, X is selected from —(CH₂)ⱼCONH(CH₂)ⱼ—, —(CH₂)ⱼ—S—S—(CH₂)ⱼ——(CH₂)ⱼNH—N═C(CH₂)ⱼ—, —(CH₂)ⱼCOO(CH₂)ⱼ—, —(CH₂)ⱼ—S—(CH₂)ⱼ—, -triazole- and a mercapto-maleimide bond;

wherein j is an integer of 0-10; preferably, j is an integer of 0-5; more preferably, j is an integer of 0-3; and in a particular embodiment of the present invention, j is 0, 1, 2, 3, 4 or 5.

Y is a linking bond between the PEG and the medicine molecule D, and the linking bond is selected from: a disulfide bond, a hydrazone bond, an amido bond, an ester bond, an ether bond, a carbonyl bond, a thioester bond or a mercapto-maleimide bond; and

preferably, Y is selected from: a disulfide bond, a hydrazone bond, an amido bond, an ester bond, a thioester bond and a mercapto-maleimide bond, wherein the disulfide bond and hydrazone bond are used for cytoplasm drug release; and the amido bond, ester bond, thioester bond and mercapto-maleimide bond are used for endonuclear drug release.

n is a quantity of branches or a quantity of arms, and n is an integer greater than or equal to 3. Preferably, n is an integer of 3-22. More preferably, n is an integer of 3-14. Most preferably, n is an integer of 3-8.

k is a quantity of branches or arms that are linked to a CPP terminal, and 1≤k≤n. Preferably, k is an integer of 1-14. More preferably, k is an integer of 1-6. In an embodiment of the present invention, k is 1, 2, 3 or 6.

In a particular embodiment of the present invention, the cell-penetrating-peptide multi-arm-PEG medicine conjugate having the general formula I has the following structures:

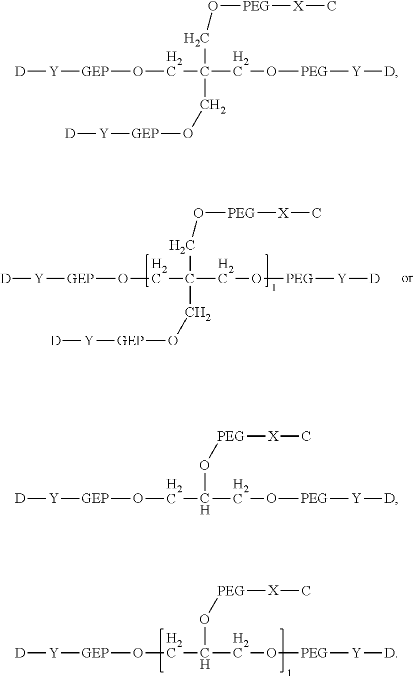

A cell-penetrating-peptide multi-arm-PEG medicine conjugate having a general formula II or III:

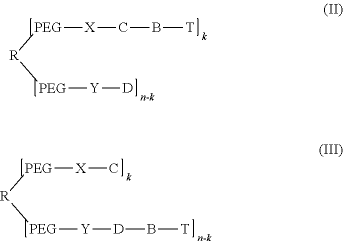

wherein the definitions of R, PEG, C, X, Y, n and k are those described in the present invention above.

D is a medicine molecule, and the medicine molecule is selected from: a small-molecule medicine, a dye, a polypeptide, a polypeptide nucleic acid, a protein, an antibody, a plasmid DNA, nucleic acid, liposome, bacteriophage particles, superparamagnetic particles, a fluorescent stain, nanoparticles, a virus, quantum dots and a magnetic-resonance-imaging contrast medium, especially siRNA, more especially Cytokine-siRNA;

preferably, D is selected from a small-molecule medicine, a polypeptide, an antibody and nucleic acid, wherein the nucleic acid includes a nucleotide monomer and an oligonucleotide; wherein the nucleotide monomer includes four deoxyribonucleotide monomers and four ribonucleotide monomers; and the oligonucleotide is a substituted oligonucleotide or an un-substituted oligonucleotide, wherein the substituted oligonucleotide is phosphorodiamidate morpholino oligomer, and the un-substituted oligonucleotide is selected from locked nucleic acid, siRNA, microRNA, an aptamer, peptide nucleic acid, decoy ODN, catalytic RNA and CpG dinucletide; and

more preferably, D is selected from siRNAs having a length of oligonucleotide of 19-23 bp.

T is a targeting group, and T is selected from: a protein, an antibody, an antibody fragment or a derivative thereof, a small-molecule peptide, a polypeptide, glucose, galactose, folic acid and hyaluronic acid; and

preferably, the antibody is a monoclonal antibody, and the antibody fragment or the derivative thereof is a single chain of an Fv or Fab fragment.

In a preferable embodiment of the present invention, T is selected from: folic acid, RGD, cRGD, hyaluronic acid, glucose and galactose.

B is a linking bond between the cell penetrating peptide or the medicine molecule and the targeting group, and the linking bond is formed by one or two or more of an amido bond, a disulfide bond, a hydrazone bond, an ester bond, a thioester bond, a mercapto-maleimide bond, a carbon-sulphur bond and an ether bond;

preferably, B is selected from one or a combination of two or more of —(CH₂)ⱼCONH(CH₂)ⱼ—, —(CH₂)ⱼ—S—S—(CH₂)ⱼ—, —(CH₂)ⱼCOO(CH₂)ⱼ—, —(CH₂)ⱼNH—N═C(CH₂)ⱼ—and —(CH₂)ⱼ—S—(CH₂)ⱼ—; and

more preferably, B is selected from —(CH₂)ⱼCONH(CH₂)ⱼ—, —(CH₂)ⱼ—S—S—(CH₂)ⱼ—, —(CH₂)ⱼCOO(CH₂)ⱼ—, —(CH₂)ⱼNH—N═C(CH₂)ⱼ— and —(CH₂)ⱼ—S—(CH₂)ⱼ—;

wherein j is an integer of 0-10; preferably, j is an integer of 0-5; more preferably, j is an integer of 0-3; and in a particular embodiment of the present invention, j is 0, 1, 2, 3, 4 or 5.

In a particular embodiment of the present invention, the cell-penetrating-peptide multi-arm-PEG medicine conjugate having the general formula II or III has the following structures:

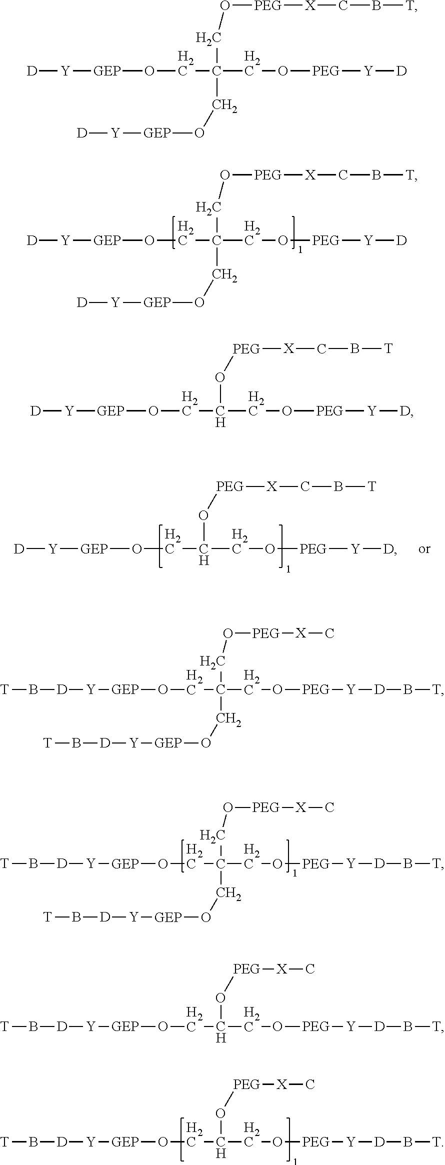

A cell-penetrating-peptide multi-arm-PEG medicine conjugate having a general formula IV:

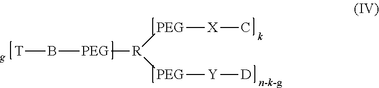

wherein R is a center molecule, and is selected from a polyhydroxy structure, a poly-amino structure or a poly-carboxyl structure;

preferably, R is selected from: a pentaerythritol or polypentaerythritol structure, a glycerol or polyglycerol structure, methyl glucoside and sucrose; and in a preferable embodiment of the present invention, R is selected from:

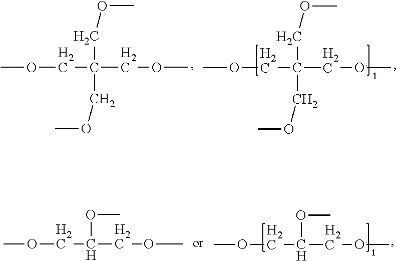

wherein l is an integer greater than or equal to 1 and smaller than or equal to 10; preferably, l is an integer greater than or equal to 1 and smaller than or equal to 10; more preferably, l is an integer greater than or equal to 1 and smaller than or equal to 7; and in a particular embodiment of the present invention, l is preferably 1, 2, 3, 4, 5 or 6.

The PEGs are the same or different —(CH₂CH₂O)ₘ—, and an average value of m is an integer of 3-250. Preferably, m is an integer of 68-250. More preferably, m is an integer of 68-227.

C is a CPP, and is selected from a transcribed trans-activator (Tat), VP22, transportan, a membrane-type amphiphilic peptide (MAP), a signal transduction peptide and an arginine-sequence-rich peptide;

preferably, C is selected from: LMWP, Tat48-60, Tat48-60-P10, CAI, HIV-TAT, MAP, MPGα, M918, R6Pen, penetratin, Pep-1-K, ARF1-22, Tp10, POD, polylysine formed by 3-100 lysine residues, and polyarginine formed by 4-9 arginine residues; and

more preferably, C is LMWP, or polyarginine formed by 8 arginines.

D is a medicine molecule, and the medicine molecule is selected from: a small-molecule medicine, a dye, a polypeptide, a polypeptide nucleic acid, a protein, an antibody, a plasmid DNA, nucleic acid, liposome, bacteriophage particles, superparamagnetic particles, a fluorescent stain, nanoparticles, a virus, quantum dots and a magnetic-resonance-imaging contrast medium, especially siRNA, more especially Cytokine-siRNA;

preferably, D is selected from a small-molecule medicine, a polypeptide, an antibody and nucleic acid, wherein the nucleic acid includes a nucleotide monomer and an oligonucleotide;

wherein the nucleotide monomer includes four deoxyribonucleotide monomers and four ribonucleotide monomers; and the oligonucleotide is a substituted oligonucleotide or an un-substituted oligonucleotide, wherein the substituted oligonucleotide is phosphorodiamidate morpholino oligomer, and the un-substituted oligonucleotide is selected from locked nucleic acid, siRNA, microRNA, an aptamer, peptide nucleic acid, decoy ODN, catalytic RNA and CpG dinucletide; and

more preferably, D is selected from a monoclonal antibody and siRNAs having a length of oligonucleotide of 19-23 bp.

T is a targeting group, and T is selected from: a protein, an antibody, an antibody fragment or a derivative thereof, a small-molecule peptide, a polypeptide, glucose, galactose, folic acid and hyaluronic acid; and

preferably, the antibody is a monoclonal antibody, and the antibody fragment or the derivative thereof is a single chain of an Fv or Fab fragment.

In a preferable embodiment of the present invention, T is selected from: folic acid, RGD, cRGD, hyaluronic acid, glucose and galactose.

X is a linking bond between the PEG and CPP, and the linking bond is formed by one or two or more of an amido bond, a disulfide bond, a hydrazone bond, an ester bond, a thioester bond, a mercapto-maleimide bond, -triazole-, a carbon-sulphur bond and an ether bond;

preferably, X is selected from one or a combination of two or more of —(CH₂)ⱼCONH(CH₂)ⱼ—, —(CH₂)ⱼ—S—S—(CH₂)ⱼ—, —(CH₂)ⱼCOO(CH₂)ⱼ—, —(CH₂)ⱼNH—N═C(CH₂)ⱼ— and —(CH₂)ⱼ—S—(CH₂)ⱼ—; and

more preferably, X is selected from —(CH₂)ⱼCONH(CH₂)ⱼ—, —(CH₂)ⱼ—S—S—(CH₂)ⱼ—, —(CH₂)ⱼCOO(CH₂)ⱼ—, —(CH₂)ⱼNH—N═C(CH₂)ⱼ— and —(CH₂)ⱼ—S—(CH₂)ⱼ—;

wherein j is an integer of 0-10; preferably, j is an integer of 0-5; more preferably, j is an integer of 0-3; and in a particular embodiment of the present invention, j is 0, 1, 2, 3, 4 or 5.

Y is a linking bond between the PEG and the medicine molecule D, and the linking bond is selected from: a disulfide bond, a hydrazone bond, an amido bond, an ester bond, an ether bond, a carbonyl bond, a thioester bond or a mercapto-maleimide bond; and

preferably, Y is selected from: a disulfide bond, a hydrazone bond, an amido bond, an ester bond, a thioester bond and a mercapto-maleimide bond, wherein the disulfide bond and hydrazone bond are used for cytoplasm drug release; and the amido bond, ester bond, thioester bond and mercapto-maleimide bond are used for endonuclear drug release.

B is a linking bond between the PEG and the targeting group, and the linking bond is formed by one or two or more of an amido bond, a disulfide bond, a hydrazone bond, an ester bond, a thioester bond, a mercapto-maleimide bond, a carbon-sulphur bond and an ether bond;

preferably, B is selected from one or a combination of two or more of —(CH₂)ⱼCONH(CH₂)ⱼ—, —(CH₂)ⱼ—S—S—(CH₂)ⱼ—, —(CH₂)ⱼCOO(CH₂)ⱼ—, —(CH₂)ⱼNH—N═C(CH₂)ⱼ— and —(CH₂)ⱼ—S—(CH₂)ⱼ—; and

more preferably, B is selected from —(CH₂)ⱼCONH(CH₂)ⱼ—, —(CH₂)ⱼ—S—S—(CH₂)ⱼ—, —(CH₂)ⱼCOO(CH₂)ⱼ—, —(CH₂)ⱼNH—N═C(CH₂)ⱼ— and —(CH₂)ⱼ—S—(CH₂)ⱼ—;

wherein j is an integer of 0-10; preferably, j is an integer of 0-5; more preferably, j is an integer of 0-3; and in a particular embodiment of the present invention, j is 0, 1, 2, 3, 4 or 5.

n is a quantity of branches or a quantity of arms, and n is an integer greater than or equal to 3. Preferably, n is an integer of 3-22. More preferably, n is an integer of 3-14. Most preferably, n is an integer of 3-8.

k is a quantity of branches or arms that are linked to a CPP terminal, and 1≤k≤n. Preferably, k is an integer of 1-14. More preferably, k is an integer of 1-6. In an embodiment of the present invention, k is 1, 2, 4 or 6.

g is a quantity of branches or arms that are linked to the targeting group, and 1≤g≤n. Preferably, g is an integer of 1-8. More preferably, g is an integer of 1-4. In an embodiment of the present invention, g is 1, 2, 3 or 4.

In a particular embodiment of the present invention, the cell-penetrating-peptide multi-arm-PEG medicine conjugate having the general formula IV has the following structures:

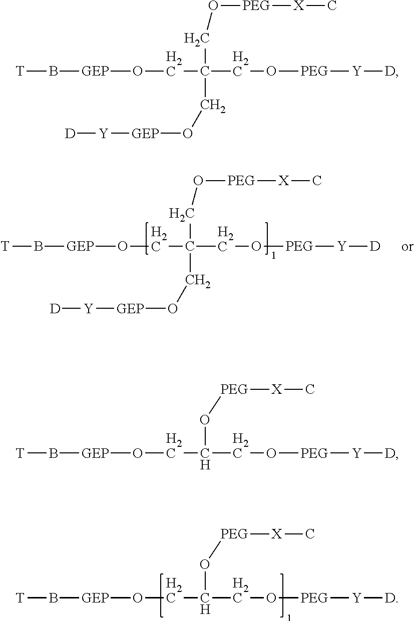

A cell-penetrating-peptide multi-arm-PEG medicine conjugate having a general formula

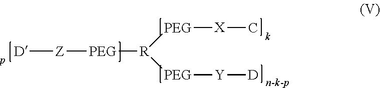

wherein R is a center molecule, and is selected from a polyhydroxy structure, a poly-amino structure or a poly-carboxyl structure;

preferably, R is selected from: a pentaerythritol or polypentaerythritol structure, a glycerol or polyglycerol structure, methyl glucoside and sucrose; and in a preferable embodiment of the present invention, R is selected from:

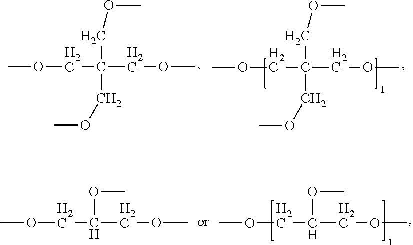

wherein l is an integer greater than or equal to 1 and smaller than or equal to 10; preferably, l is an integer greater than or equal to 1 and smaller than or equal to 10; more preferably, l is an integer greater than or equal to 1 and smaller than or equal to 7; and in a particular embodiment of the present invention, l is preferably 1, 2, 3, 4, 5 or 6.

The PEGs are the same or different —(CH₂CH₂O)ₘ—, and an average value of m is an integer of 3-250. Preferably, m is an integer of 68-250. More preferably, m is an integer of 68-227.

C is a CPP, and is selected from a transcribed trans-activator (Tat), VP22, transportan, a membrane-type amphiphilic peptide (MAP), a signal transduction peptide and an arginine-sequence-rich peptide;

preferably, C is selected from: LMWP, Tat48-60, Tat48-60-P10, CAI, HIV-TAT, MAP, MPGα, M918, R6Pen, penetratin, Pep-1-K, ARF1-22, Tp10, POD, polylysine formed by 3-100 lysine residues, and polyarginine formed by 4-9 arginine residues; and

more preferably, C is LMWP, or polyarginine formed by 8 arginines.

D and D′ are independently selected from: a small-molecule medicine, a dye, a polypeptide, polypeptide nucleic acid, a protein, an antibody, a plasmid DNA, nucleic acid, liposome, bacteriophage particles, superparamagnetic particles, a fluorescent stain, nanoparticles, a virus, quantum dots and a magnetic-resonance-imaging contrast medium, especially siRNA, more especially Cytokine-siRNA;

preferably, D is selected from a small-molecule medicine; more preferably, D is selected from a small-molecule medicine, wherein the small-molecule medicine has a molecular weight less than 1000, such as vitamin C, acetylsalicylic acid, acetaminophen and paracetamol;

preferably, D′ is selected from nucleic acid, wherein the nucleic acid includes a nucleotide monomer and an oligonucleotide; wherein the nucleotide monomer includes four deoxyribonucleotide monomers and four ribonucleotide monomers; and the oligonucleotide is a substituted oligonucleotide or an un-substituted oligonucleotide, wherein the substituted oligonucleotide is phosphorodiamidate morpholino oligomer, and the un-substituted oligonucleotide is selected from locked nucleic acid, siRNA, microRNA, an aptamer, peptide nucleic acid, decoy ODN, catalytic RNA and CpG dinucletide; and

more preferably, D′ is selected from a monoclonal antibody and siRNAs having a length of oligonucleotide of 19-23 bp.

X is a linking bond between the PEG and CPP, and the linking bond is formed by one or two or more of an amido bond, a disulfide bond, a hydrazone bond, an ester bond, a thioester bond, a mercapto-maleimide bond, -triazole-, a carbon-sulphur bond and an ether bond;

preferably, X is selected from one or a combination of two or more of —(CH₂)ⱼCONH(CH₂)ⱼ—, —(CH₂)ⱼ—S—S—(CH₂)ⱼ—, —(CH₂)ⱼCOO(CH₂)ⱼ—, —(CH₂)ⱼNH—N═C(CH₂)ⱼ— and —(CH₂)ⱼ—S—(CH₂)ⱼ—; and

more preferably, X is selected from —(CH₂)ⱼCONH(CH₂)ⱼ—, —(CH₂)ⱼ—S—S—(CH₂)ⱼ—, —(CH₂)ⱼCOO(CH₂)ⱼ—, —(CH₂)ⱼNH—N═C(CH₂)ⱼ— and —(CH₂)ⱼ—S—(CH₂)—;

wherein j is an integer of 0-10; preferably, j is an integer of 0-5; more preferably, j is an integer of 0-3; and in a particular embodiment of the present invention, j is 0, 1, 2, 3, 4 or 5.

Y is a linking bond between the PEG and the medicine molecule D, and the linking bond is selected from: a disulfide bond, a hydrazone bond, an amido bond, an ester bond, an ether bond, a carbonyl bond, a thioester bond or a mercapto-maleimide bond; and

preferably, Y is selected from: a disulfide bond, a hydrazone bond, an amido bond, an ester bond, a thioester bond and a mercapto-maleimide bond, wherein the disulfide bond and hydrazone bond are used for cytoplasm drug release; and the amido bond, ester bond, thioester bond and mercapto-maleimide bond are used for endonuclear drug release.

Z is a linking bond between the PEG and the medicine molecule D′, and the linking bond is formed by one or two or more of an amido bond, a disulfide bond, a hydrazone bond, an ester bond, a thioester bond, a mercapto-maleimide bond, a carbon-sulphur bond and an ether bond;

preferably, Z is selected from one or a combination of two or more of —(CH₂)ⱼCONH(CH₂)ⱼ—, —(CH₂)ⱼ—S—S—(CH₂)ⱼ—, —(CH₂)ⱼCOO(CH₂)ⱼ—, —(CH₂)ⱼNH—N═C(CH₂)ⱼ— and —(CH₂)ⱼ—S—(CH₂)ⱼ—; and

more preferably, Z is selected from —(CH₂)ⱼCONH(CH₂)ⱼ—, —(CH₂)ⱼ—S—S—(CH₂)ⱼ—, —(CH₂)ⱼCOO(CH₂)ⱼ—, —(CH₂)ⱼNH—N═C(CH₂)ⱼ— and —(CH₂)ⱼ—S—(CH₂)ⱼ—;

wherein j is an integer of 0-10; preferably, j is an integer of 0-5; more preferably, j is an integer of 0-3; and in a particular embodiment of the present invention, j is 0, 1, 2, 3, 4 or 5.

n is a quantity of branches or a quantity of arms, and n is an integer greater than or equal to 3. Preferably, n is an integer of 3-22. More preferably, n is an integer of 3-14. Most preferably, n is an integer of 3-8.

k is a quantity of branches or arms that are linked to a CPP terminal, and 1≤k≤n. Preferably, k is an integer of 1-14. More preferably, k is an integer of 1-6. In an embodiment of the present invention, k is 1, 2, 4 or 6.

p is a quantity of branches or arms that are linked to the medicine molecule D′, and 1≤p≤n. Preferably, and p is an integer of 1-8. More preferably, and p is an integer of 1-4. In an embodiment of the present invention, p is 1, 2, 3 or 4.

In a particular embodiment of the present invention, the cell-penetrating-peptide multi-arm-PEG medicine conjugate having the general formula V has the following structures:

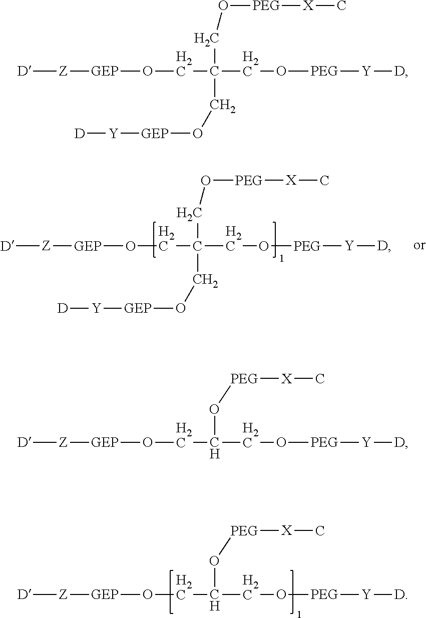

In the present invention, the molecular weight of the PEG is 1000-80000 Da. Preferably, the molecular weight of the PEG is 3000-20000 Da. More preferably, the molecular weight of the PEG is 3000-10000 Da. In a most preferable embodiment of the present invention, the molecular weight of the PEG may be 3000 Da, 5000 Da, 10000 Da or 20000 Da.

The present invention further provides a medicine composition, wherein the medicine composition includes the cell-penetrating-peptide multi-arm-PEG medicine conjugate having the general formula I, II, III, IV or V.

Preferably, the medicine composition further includes one or more pharmaceutically acceptable excipients, wherein the excipients are selected from: a carrier, a diluent, a binder, a lubricant and a wetting agent.

Preferably, the dosage forms of the medicine composition include: tablets, capsules, pills, an injection, an emulsion, a microemulsion, nanoparticles, an inhalant, a lozenge, gel, powder, a suppository, a suspension emulsion, cream, jelly and a spray.

Preferably, the feasible administration modes of the medicine composition include: oral administration, subcutaneous injection, intramuscular injection, intravenous injection, rectal administration, vaginal administration, intranasal administration, transdermal administration, subconjunctival administration, intraocular administration, orbital administration, retrobulbar administration, retinal administration, choroid administration and intrathecal injection.

The use of the cell-penetrating-peptide multi-arm-PEG medicine conjugate having the general formula I, II, III, IV or V according to the present invention in preparation of a targeting medicine, wherein the medicine is used for diagnosis and treatment of a disease. Preferably, and the disease is selected from tumor, pneumonia, asthma, pulmonary fibrosis, virus infection, hepatitis, and eye age-related macular degeneration. Preferably, the disease is selected from eye age-related macular degeneration, asthma and pulmonary fibrosis.

In the cell-penetrating-peptide multi-arm-PEG medicine conjugate having the general formula I, II, III/IV or V according to the present invention, as compared with linear-chain-type PEG-cell-penetrating-peptide conjugates, the multi-arm PEG has multiple end groups, and has introduction sites for multiple functional groups, which can connect to multiple different active groups, which prevents the problems of linear-chain-type PEGs on limited linkage sites, a small scope of application and a low medicine loading capacity. In addition, the cell-penetrating-peptide multi-arm-PEG medicine conjugate according to the present invention has targeting ability, and according to the particular demands of treatment, targeting groups can be connected to the cell penetrating peptide, the medicine molecule or the PEG chain ends, to enable the medicine to enter the pathogenetic cells in a targeting manner, to achieve precise treatment.

The amino-acid sequences of the CPPs according to the present invention are as follows:

LMWP
VSRRRRRRGGRRRR
Tat48-60
GRKKRRQRRRPPQ
Tat48-60-P10
GRKKRRQRRRPPQRQTSMTDFYHSKRRLIFS
CAI
ITFEDLLDYYGP-NH₂
HIV-TAT
RKKRRQRRR
MAP
KLALKLALKALKAALKLA-NH₂
MPGα
Ac-GALFLAFLAAALSCMGLWSQPKKKRKV-Cya
M918
MVTVLFRRLRIRRACGPPRVRV-NH₂
R6Pen
RRRRRRRQIKIWFQNRRMKWKK
Penetratin
RQIKIWFQNRRMKWKK
Pep-1-K
KKTWWKTWWTKWSQPKKKRKV
ARF1-22
MVRRFLVTLRIRRACGPPRVRV-NH₂
Tp10
AGYLLGKINLKALAALAKKIL-NH₂
POD
GGG(ARKKAAKA)4

### DETAILED DESCRIPTION OF THE EMBODIMENTS

The technical solutions of the present invention will be described clearly and completely below with reference to the embodiments of the present invention. Apparently, the described embodiments are merely certain embodiments of the present invention, rather than all of the embodiments. All of the other embodiments that a person skilled in the art obtains on the basis of the embodiments in the present invention without paying creative work fall within the protection scope of the present invention.

### Example 1

### Preparation Method of Four-Arm PEG-LMWP Conjugate (MW10000)

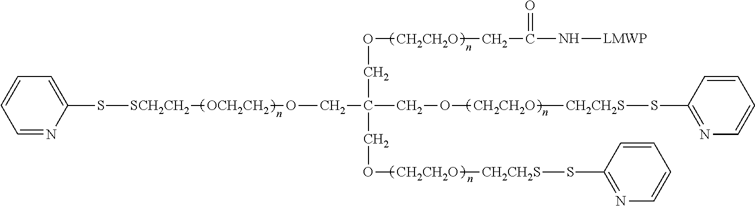

LMWP was dissolved in 20 mM of a KH₂PO₄ buffer solution. 4arm PEG-1 arm NHS-3arm Opss was dissolved in DMSO, and then added dropwise into the buffer solution. The reaction was performed at room temperature for 2 hours. The product was filtered, purified by using a heparin column, and freeze-dried or precipitated.

### Example 2

### Preparation Method of Four-Arm PEG-LMWP-MAL Conjugate (MW10000)

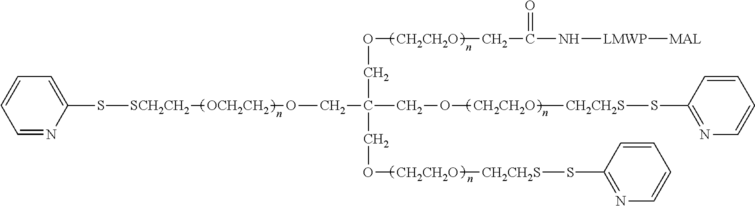

The above-prepared 4arm PEG-1 arm LMWP-3arm OPSS was dissolved in dichloromethane. Proper amounts of TEA and MAL-NH2 were added. The reaction was performed at room temperature for 12 hours. The reaction liquor was concentrated. The product was purified and freeze-dried or precipitated by using isopropanol.

### Example 3

### Preparation Method of Four-Arm PEG-LMWP-MAL-RGD Conjugate (MW10000)

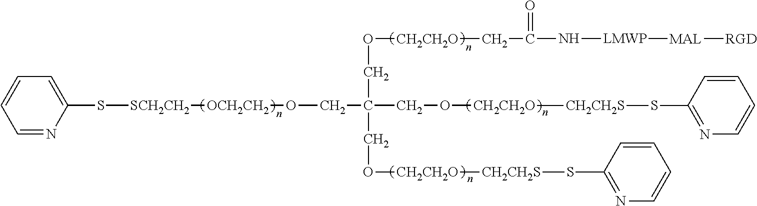

The above-prepared 4arm PEG-1 arm LMWP-MAL-3arm OPSS was dissolved in dichloromethane. A proper amount of RGD was added. The reaction was performed at room temperature for 12 hours. The reaction liquor was concentrated. The product was purified and freeze-dried or precipitated by using isopropanol.

### Example 4

### Preparation Method of Four-Arm PEG-LMWP-MAL-RGD-3arm-VEGF-siRNA Conjugate (MW10000)

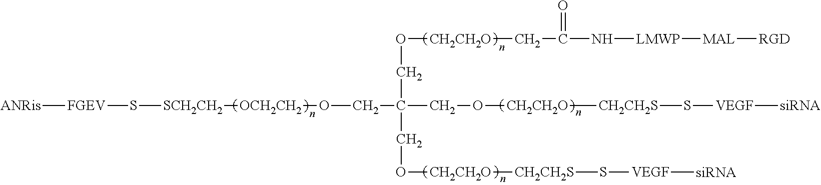

A 1M DTT solution was added into the above-prepared 4arm PEG-1arm LMWP-MAL-RGD-3 arm OPSS. The reaction was performed at room temperature, to obtain 4arm PEG-1arm LMWP-MAL-RGD-3arm Thiol. Then a mutant SH-VEGF-siRNA was dissolved in 10 mM KH₂PO₄ and 0.15M NaCl, and added dropwise into a proper amount of 4arm PEG-1 arm LMWP-MAL-RGD-3arm Thiol purified by using a heparin column under stirring. The mixture was reacted under continuous stirring for 2 hours. After the reaction has completed, the unreacted SH-VEGF-siRNA was removed by cation-column purification. The product was obtained by freeze drying or precipitation, and was stored at −20° C.

The sequences of the positive-sense strand and the antisense strand of the SH-VEGF-siRNA are as follows:

Positive-sense strand: 5′-GAUAGAGCAAGACAAGAAAUU-3′
Antisense strand: 3′-UUCUAUCUCGUUCUGUUCUUU-5′

### Example 5

### Preparation Method of Four-Arm PEG-LMWP-(VEGF-siRNA)3 Conjugate (MW10000)

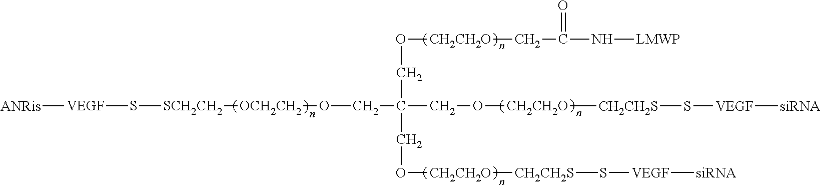

The above-prepared 4arm PEG-1 arm LMWP-3arm OPSS solution was directly added dropwise into a 20 mM NaH₂PO₄+1 mM EDTA buffer solution of pH=6.9 containing a certain amount of SH-VEGF-siRNA under stirring. The system was reacted at 40° C. for 30 min to 1 h, and when detection by using gel electrophoresis showed that the SH-VEGF-siRNA has completely reacted, the reaction was stopped. The reaction liquor was purified by using a DEAE column, wherein the system of the mobile phase was formed by a phase A: a 20 mM NaH₂PO₄+1 mM EDTA buffer solution of PH=6.9, and a phase B: a 20 mM NaH₂PO₄+2M NaCl+1 mM EDTA buffer solution of PH=6.9. The sample was collected at 45% of the phase B, and the gel electrophoresis shown that the product was of a strip. The sample was desalted by using an ultrafiltration centrifuge tube with a cut-off molecular weight of 3000. The product was obtained by freeze drying or precipitation, and was stored at −20° C.

### Example 6

### Preparation Method of Four-Arm PEG-(LMWP)2 Conjugate (MW10000)

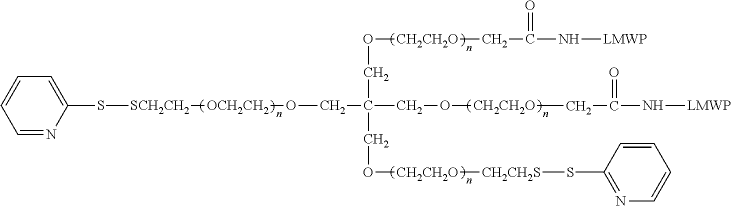

LMWP was dissolved in a 20 mM KH₂PO₄ buffer solution. 4arm PEG-2arm NHS-2arm Opss was dissolved in DMSO, and then added dropwise into the buffer solution. The reaction was performed at room temperature for 2 hours. The product was filtered, purified by using a heparin column, and freeze-dried. The solution may also be directly used for the next step of reaction.

### Example 7

### Preparation Method of Four-Arm PEG-(LMWP)2-(VEGF-siRNA)2 Conjugate (MW10000)

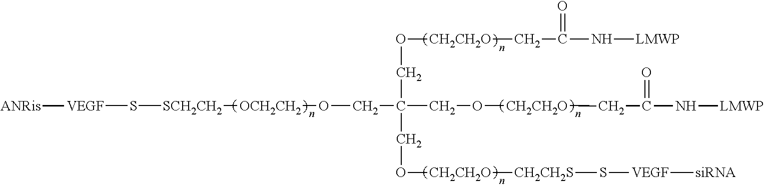

The above-prepared 4arm PEG-2arm LMWP-2arm OPSS solution was directly added dropwise into a 20 mM NaH₂PO₄+1 mM EDTA buffer solution of pH=6.9 containing a certain amount of SH-VEGF-siRNA under stirring. The system was reacted at 40° C. for 30 min to 1 h, and when detection by using gel electrophoresis showed that the SH-VEGF-siRNA has completely reacted, the reaction was stopped. The reaction liquor was purified by using a DEAE column, wherein the system of the mobile phase was formed by a phase A: a 20 mM NaH₂PO₄+1 mM EDTA buffer solution of PH=6.9, and a phase B: a 20 mM NaH₂PO₄+2M NaCl+1 mM EDTA buffer solution of PH=6.9. The sample was collected at 45% of the phase B, and the gel electrophoresis shown that the product was of a strip. The sample was desalted by using an ultrafiltration centrifuge tube with a cut-off molecular weight of 3000. The product was obtained by freeze drying or precipitation, and was stored at −20° C.

### Example 8

### Preparation Method of Four-Arm PEG-(LMWP)3 Conjugate (MW10000)

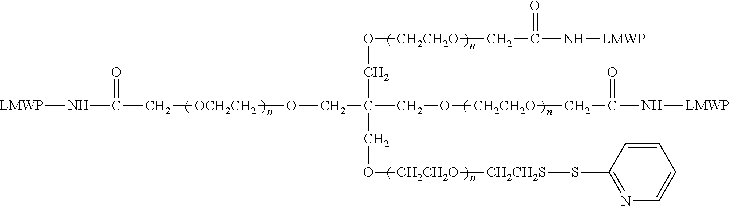

LMWP was dissolved in a 20 mM KH₂PO₄ buffer solution. 4arm PEG-3arm NHS-1arm Opss was dissolved in DMSO, and then added dropwise into the buffer solution. The reaction was performed at room temperature for 2 hours. The product was filtered, purified by using a heparin column, and freeze-dried. The solution may also be directly used for the next step of reaction.

### Example 9

### Preparation Method of Four-Arm PEG-(LMWP)3-VEGF-siRNA Conjugate (MW10000)

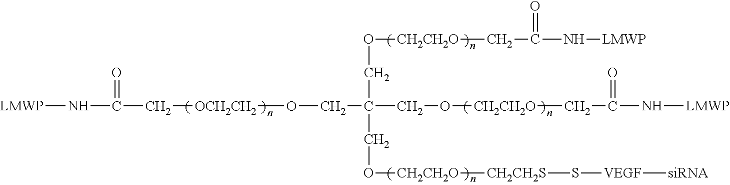

The above-prepared 4arm PEG-3 arm LMWP-1arm OPSS solution was directly added dropwise into a 20 mM NaH₂PO₄+1 mM EDTA buffer solution of pH=6.9 containing a certain amount of SH-VEGF-siRNA under stirring. The system was reacted at 40° C. for 30 min to 1 h, and when detection by using gel electrophoresis showed that the SH-VEGF-siRNA has completely reacted, the reaction was stopped. The reaction liquor was purified by using a DEAE column, wherein the system of the mobile phase was formed by a phase A: a 20 mM NaH₂PO₄+1 mM EDTA buffer solution of PH=6.9, and a phase B: a 20 mM NaH₂PO₄+2 M NaCl+1 mM EDTA buffer solution of PH=6.9. The sample was collected at 45% of the phase B, and the gel electrophoresis shown that the product was of a strip. The sample was desalted by using an ultrafiltration centrifuge tube with a cut-off molecular weight of 3000. The product was obtained by freeze drying or precipitation, and was stored at −20° C.

### Example 10

### Preparation Method of Eight-Arm PEG-LMWP Conjugate (MW10000)

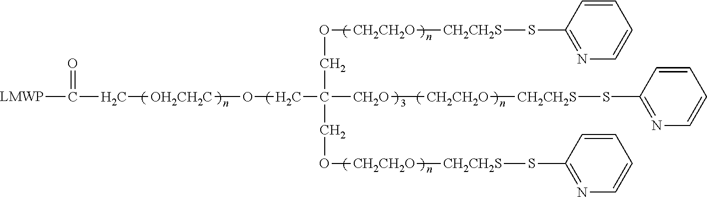

LMWP was dissolved in a 20 mM KH₂PO₄ buffer solution. 8arm PEG-1arm NHS-7arm Opss was dissolved in DMSO, and then added dropwise into the buffer solution. The reaction was performed at room temperature for 2 hours. The product was filtered, purified by using a heparin column, and freeze-dried or precipitated.

### Example 11

### Preparation Method of Eight-Arm PEG-(LMWP)1-(VEGF-siRNA)7 Conjugate (MW10000)

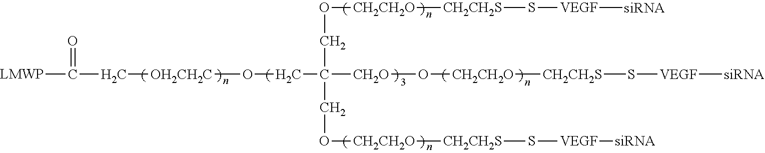

The above-prepared 8arm PEG-1arm LMWP-7arm OPSS solution was directly added dropwise into a 20mM NaH₂PO₄+1 mM EDTA buffer solution of pH=6.9 containing a certain amount of

SH-VEGF-siRNA under stirring. The system was reacted at 40° C. for 30 min to 1 h, and when detection by using gel electrophoresis showed that the SH-VEGF-siRNA has completely reacted, the reaction was stopped. The reaction liquor was purified by using a DEAE column, wherein the system of the mobile phase was formed by a phase A: a 20 mM NaH₂PO₄+1 mM EDTA buffer solution of PH=6.9, and a phase B: a 20 mM NaH₂PO₄+2M NaCl+1 mM EDTA buffer solution of PH=6.9. The sample was collected at 45% of the phase B, and the gel electrophoresis shown that the product was of a strip. The sample was desalted by using an ultrafiltration centrifuge tube with a cut-off molecular weight of 3000. The product was obtained by freeze drying or precipitation, and was stored at −20° C.

### Example 12

### Preparation Method of Eight-Arm PEG-(LMWP)2 Conjugate (MW10000)

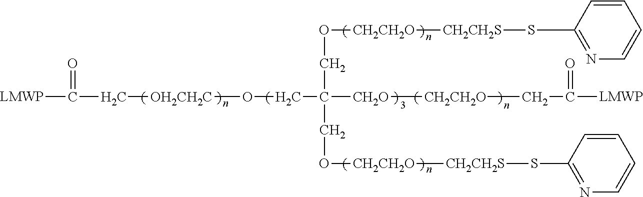

LMWP was dissolved in a 20 mM KH₂PO₄ buffer solution. 8arm PEG-2arm NHS-6arm Opss was dissolved in DMSO, and then added dropwise into the buffer solution. The reaction was performed at room temperature for 2 hours. The product was filtered, purified by using a heparin column, and freeze-dried or precipitated.

### Example 13

### Preparation Method of Eight-Arm PEG-(LMWP)2-(VEGF-siRNA)6 Conjugate (MW10000)

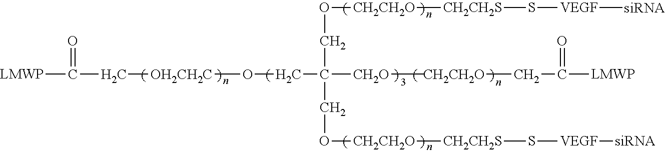

The above-prepared 8arm PEG-2arm LMWP-6arm OPSS solution was directly added dropwise into a 20 mM NaH₂PO₄+1 mM EDTA buffer solution of pH=6.9 containing a certain amount of SH-VEGF-siRNA under stirring. The system was reacted at 40° C. for 30 min to 1 h, and when detection by using gel electrophoresis showed that the SH-VEGF-siRNA has completely reacted, the reaction was stopped. The reaction liquor was purified by using a DEAE column, wherein the system of the mobile phase was formed by a phase A: a 20 mM NaH₂PO₄+1 mM EDTA buffer solution of PH=6.9, and a phase B: a 20 mM NaH₂PO₄+2M NaCl+1 mM EDTA buffer solution of PH=6.9. The sample was collected at 45% of the phase B, and the gel electrophoresis shown that the product was of a strip. The sample was desalted by using an ultrafiltration centrifuge tube with a cut-off molecular weight of 3000. The product was obtained by freeze drying or precipitation, and was stored at −20° C.

### Example 14

### Comparison of the Cell Effect Inhibitions Between LMWP-PEG5000-VEGF-siRNA and the Four-Arm PEG-LMWP-(VEGF-siRNA)3 (MW 10000)

Cell transfection was performed on LMWP-PEG5000-VEGF-siRNA and the four-arm PEG-LMWP-(VEGF-siRNA)₃ (MW 10000) prepared in Example 5 on 293T cell, and the expression of the VEGFA in the sample was detected by using RT-PCR. The result showed that the cell effect of the four-arm PEG-LMWP-(VEGF-siRNA)₃ (MW 10000) is obviously better than that of LMWP-PEG5000-VEGF-siRNA.

The above particular embodiments are merely illustrative explain on the present invention, and are not limiting the present invention. A person skilled in the art can understand that the particular structures in the present invention may have other variations.

1. A cell-penetrating-peptide multi-arm-PEG medicine conjugate having a general formula I: 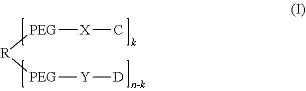
wherein R is a center molecule, and is selected from a polyhydroxy structure, a poly-amino structure or a poly-carboxyl structure;
the PEGs are the same or different —(CH₂CH₂O)ₘ—, and an average value of m is an integer of 3-250;
C is a cell penetrating peptide (CPP), and is selected from a transcribed trans-activator, VP22, transportan, a membrane-type amphiphilic peptide, a signal transduction peptide and an arginine-sequence-rich peptide;
D is a medicine molecule, and the medicine molecule is selected from: a small-molecule medicine, a dye, a polypeptide, an antibody, a plasmid DNA, nucleic acid, liposome, bacteriophage particles, superparamagnetic particles, a fluorescent stain, nanoparticles, a virus, quantum dots and a magnetic-resonance-imaging contrast medium;
X is a linking bond between the PEG and CPP, and the linking bond is formed by one or two or more of an amido bond, a disulfide bond, a hydrazone bond, an ester bond, a thioester bond, a mercapto-maleimide bond, --triazole-, a carbon-sulphur bond and an ether bond;
Y is a linking bond between the PEG and the medicine molecule D, and the linking bond is selected from: a disulfide bond, a hydrazone bond, an amido bond, an ester bond, an ether bond, a carbonyl bond, a thioester bond or a mercapto-maleimide bond; and
n is a quantity of branches or a quantity of arms, and n is an integer greater than or equal to 3; and k is a quantity of branches or arms that are linked to a CPP terminal, and 1≤k≤n.

2. The cell-penetrating-peptide multi-arm-PEG medicine conjugate according to claim 1, characterized in that R is selected from: a pentaerythritol or polypentaerythritol structure, a glycerol or polyglycerol structure, methyl glucoside and sucrose;
C is selected from: LMWP, Tat48-60, Tat48-60-P10, CAI, HIV-TAT, MAP, MPGα, M918, R6Pen, penetratin, Pep-1-K, ARF1-22, Tp10, POD, polylysine formed by 3-100 lysine residues, and polyarginine formed by 4-9 arginine residues;
D is selected from a small-molecule medicine, a polypeptide, an antibody and nucleic acid;
X is selected from one or a combination of two or more of —(CH₂)ⱼCONH(CH₂)ⱼ—, —(CH₂)ⱼ—S—S—(CH₂)ⱼ—, —(CH₂)ⱼNH—N═C(CH₂)ⱼ—, —(CH₂)ⱼCOO(CH₂)ⱼ—, —(CH₂)ⱼ—S—(CH₂)ⱼ—, -triazole- and a mercapto-maleimide bond, wherein j is an integer of 0-10;
Y is selected from: a disulfide bond, a hydrazone bond, an amido bond, an ester bond, a thioester bond and a mercapto-maleimide bond; and
n is an integer of 3-22; and k is an integer of 1-14.

3. The cell-penetrating-peptide multi-arm-PEG medicine conjugate according to claim 2, characterized in that R is selected from: 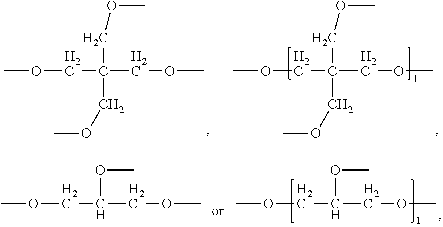
wherein l is an integer greater than or equal to 1 and smaller than or equal to 10;
C is LMWP, or polyarginine formed by 8 arginines;
D is selected from Omalizumab, Nintedanib, Bevacizumab, Pembrolizumab, Trastuzumab, Nivolumab, VEGF-siRNA, IL-v-siRNA, Syk-siRNA and GATA-3-siRNA, wherein v is selected from 4, 5, 8 and 13;
X is selected from —(CH₂)ⱼCONH(CH₂)ⱼ—, —(CH₂)ⱼ—S—S—(CH₂)ⱼ—, —(CH₂)ⱼNH—N═C(CH₂)ⱼ—, —(CH₂)ⱼCOO(CH₂)ⱼ—, —(CH₂)ⱼ—S—(CH₂)ⱼ—, -triazole- and a mercapto-maleimide bond, wherein j is an integer of 0-5; and
n is an integer of 3-14; and k is an integer of 1-6.

4. The cell-penetrating-peptide multi-arm-PEG medicine conjugate having a general formula I according to claim 1, having a structure of:

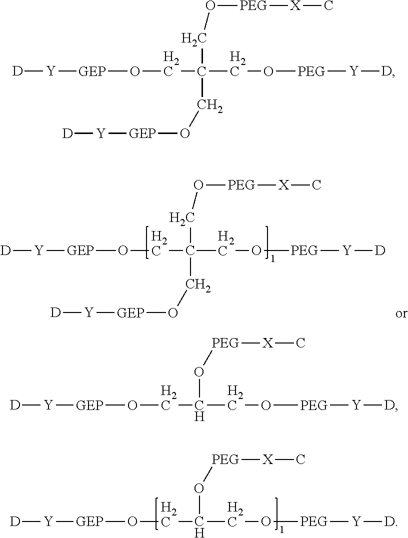

5-8. (canceled)

9. A cell-penetrating-peptide multi-arm-PEG medicine conjugate having a general formula IV: 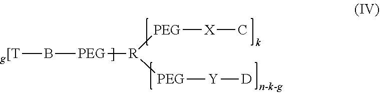
wherein R is a center molecule, and is selected from a polyhydroxy structure, a poly-amino structure or a poly-carboxyl structure;
the PEGs are the same or different —(CH₂CH₂O)ₘ—, and an average value of m is an integer of 3-250;
C is a CPP, and is selected from a transcribed trans-activator, VP22, transportan, a membrane-type amphiphilic peptide, a signal transduction peptide and an arginine-sequence-rich peptide;
D is a medicine molecule, and the medicine molecule is selected from: a small-molecule medicine, a dye, a polypeptide, an antibody, a plasmid DNA, nucleic acid, liposome, bacteriophage particles, superparamagnetic particles, a fluorescent stain, nanoparticles, a virus, quantum dots and a magnetic-resonance-imaging contrast medium;
T is a targeting group, and T is selected from: a protein, an antibody, an antibody fragment or a derivative thereof, a small-molecule peptide, a polypeptide, glucose, galactose, folic acid and hyaluronic acid;
X is a linking bond between the PEG and CPP, and the linking bond is formed by one or two or more of an amido bond, a disulfide bond, a hydrazone bond, an ester bond, a thioester bond, a mercapto-maleimide bond, -triazole-, a carbon-sulphur bond and an ether bond;
Y is a linking bond between the PEG and the medicine molecule D, and the linking bond is selected from: a disulfide bond, a hydrazone bond, an amido bond, an ester bond, an ether bond, a carbonyl bond, a thioester bond or a mercapto-maleimide bond;
B is a linking bond between the PEG and the targeting group, and the linking bond is formed by one or two or more of an amido bond, a disulfide bond, a hydrazone bond, an ester bond, a thioester bond, a mercapto-maleimide bond, a carbon-sulphur bond and an ether bond; and
n is a quantity of branches or a quantity of arms, and n is an integer greater than or equal to 3; k is a quantity of branches or arms that are linked to a CPP terminal, and 1≤k≤n; and g is a quantity of branches or arms that are linked to the targeting group, and 1≤g≤n.

10. The cell-penetrating-peptide multi-arm-PEG medicine conjugate according to claim 9, characterized in that R is selected from: a pentaerythritol or polypentaerythritol structure, a glycerol or polyglycerol structure, methyl glucoside and sucrose;
C is selected from: LMWP, Tat48-60, Tat48-60-P10, CAI, HIV-TAT, MAP, MPGα, M918, R6Pen, penetratin, Pep-1-K, ARF1-22, Tp10, POD, polylysine formed by 3-100 lysine residues, and polyarginine formed by 4-9 arginine residues;
D is selected from a small-molecule medicine, a polypeptide, an antibody and nucleic acid;
the antibody in T is a monoclonal antibody, and the antibody fragment or the derivative thereof is a single chain of an Fv or Fab fragment;
X is selected from one or a combination of two or more of —(CH₂)ⱼCONH(CH₂)ⱼ—, —(CH₂)ⱼ—S—S—(CH₂)ⱼ—, —(CH₂)ⱼNH—N═C(CH₂)ⱼ—, —(CH₂)ⱼCOO(CH₂)ⱼ— and —(CH₂)ⱼ—S—(CH₂)ⱼ—, wherein j is an integer of 0-10;
Y is selected from: a disulfide bond, a hydrazone bond, an amido bond, an ester bond, a thioester bond and a mercapto-maleimide bond;
B is selected from one or a combination of two or more of —(CH₂)ⱼCONH(CH₂)ⱼ—, —(CH₂)ⱼ—S—S—(CH₂)ⱼ—, —(CH₂)ⱼNH—N═C(CH₂)ⱼ—, —(CH₂)ⱼCOO(CH₂)ⱼ— and —(CH₂)ⱼ—S—(CH₂)ⱼ—, wherein j is an integer of 0-10; and
n is an integer of 3-22; k is an integer of 1-14; and g is an integer of 1-8.

11. The cell-penetrating-peptide multi-arm-PEG medicine conjugate according to claim 10, characterized in that R is selected from: 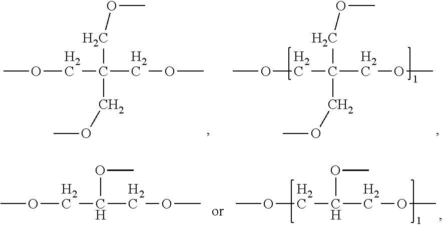
wherein l is an integer greater than or equal to 1 and smaller than or equal to 10;
C is LMWP, or polyarginine formed by 8 arginines;
D is selected from a monoclonal antibody and siRNAs having a length of oligonucleotide of 19-23 bp;
T is selected from: folic acid, RGD, cRGD, hyaluronic acid, glucose and galactose;
X is selected from —(CH₂)ⱼCONH(CH₂)ⱼ—, —(CH₂)ⱼ—S—S—(CH₂)ⱼ—, —(CH₂)ⱼNH—N═C(CH₂)ⱼ—, —(CH₂)ⱼCOO(CH₂)ⱼ— and —(CH₂)ⱼ—S—(CH₂)ⱼ—, wherein j is an integer of 0-5;
B is selected from —CH₂)ⱼCONH(CH₂)ⱼ—, —(CH₂)ⱼ—S—S—(CH₂)ⱼ—, —(CH₂)ⱼNH—N═C(CH₂)ⱼ—, —(CH₂)ⱼCOO(CH₂)ⱼ— and —(CH₂)ⱼ—S—(CH₂)ⱼ—, wherein j is an integer of 0-5; and
n is an integer of 3-14; k is an integer of 1-6; and g is an integer of 1-4.

12. The cell-penetrating-peptide multi-arm-PEG medicine conjugate having a general formula IV according to claim 9, having a structure of:

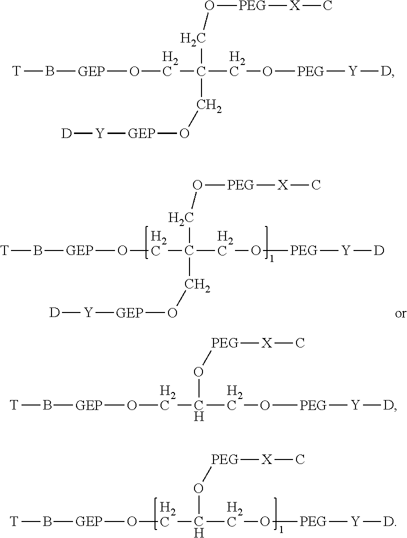

13. A cell-penetrating-peptide multi-arm-PEG medicine conjugate having a general formula V: 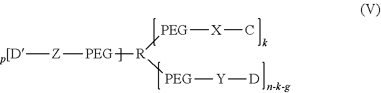
wherein R is a center molecule, and is selected from a polyhydroxy structure, a poly-amino structure or a poly-carboxyl structure;
the PEGs are the same or different —(CH₂CH₂O)ₘ—, and an average value of m is an integer of 3-250;
C is a CPP, and is selected from a transcribed trans-activator (Tat), VP22, transportan, a membrane-type amphiphilic peptide (MAP), a signal transduction peptide and an arginine-sequence-rich peptide;
D and D′ are independently selected from: a small-molecule medicine, a dye, a polypeptide, an antibody, a plasmid DNA, nucleic acid, liposome, bacteriophage particles, superparamagnetic particles, a fluorescent stain, nanoparticles, a virus, quantum dots and a magnetic-resonance-imaging contrast medium;
X is a linking bond between the PEG and CPP, and the linking bond is formed by one or two or more of an amido bond, a disulfide bond, a hydrazone bond, an ester bond, a thioester bond, a mercapto-maleimide bond, -triazole-, a carbon-sulphur bond and an ether bond;
Y is a linking bond between the PEG and the medicine molecule D, and the linking bond is selected from: a disulfide bond, a hydrazone bond, an amido bond, an ester bond, an ether bond, a carbonyl bond, a thioester bond or a mercapto-maleimide bond;
Z is a linking bond between the PEG and the medicine molecule D′, and the linking bond is formed by one or two or more of an amido bond, a disulfide bond, a hydrazone bond, an ester bond, a thioester bond, a mercapto-maleimide bond, a carbon-sulphur bond and an ether bond; and
n is a quantity of branches or a quantity of arms, and n is an integer greater than or equal to 3; k is a quantity of branches or arms that are linked to a CPP terminal, and 1≤k≤n; and p is a quantity of branches or arms that are linked to the medicine molecule D′, and 1≤p≤n.

14. The cell-penetrating-peptide multi-arm-PEG medicine conjugate according to claim 13, characterized in that R is selected from: a pentaerythritol or polypentaerythritol structure, a glycerol or polyglycerol structure, methyl glucoside and sucrose;
C is selected from: LMWP, Tat48-60, Tat48-60-P10, CAI, HIV-TAT, MAP, MPGα, M918, R6Pen, penetratin, Pep-1-K, ARF1-22, Tp10, POD, polylysine formed by 3-100 lysine residues, and polyarginine formed by 4-9 arginine residues;
D is selected from a small-molecule medicine;
D′ is selected from nucleic acid;
X is selected from one or a combination of two or more of —(CH₂)ⱼCONH(CH₂)ⱼ—, —(CH₂)ⱼ—S—S—(CH₂)ⱼ—, —(CH₂)ⱼNH—N═C(CH₂)ⱼ—, —(CH₂)ⱼCOO(CH₂)ⱼ— and —(CH₂)ⱼ—S—(CH₂)ⱼ—, wherein j is an integer of 0-10;
Y is selected from: a disulfide bond, a hydrazone bond, an amido bond, an ester bond, a thioester bond and a mercapto-maleimide bond;
Z is selected from one or a combination of two or more of —(CH₂)ⱼCONH(CH₂)ⱼ—, —(CH₂)ⱼ—S—S—(CH₂)ⱼ—, —(CH₂)ⱼNH—N═C(CH₂)ⱼ—, —(CH₂)ⱼCOO(CH₂)ⱼ— and —(CH₂)ⱼ—S—(CH₂)ⱼ—, wherein j is an integer of 0-10; and
n is an integer of 3-22; k is an integer of 1-14; and p is an integer of 1-8.

15. The cell-penetrating-peptide multi-arm-PEG medicine conjugate according to claim 13, characterized in that R is selected from: 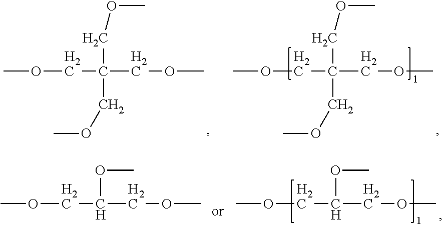
wherein l is an integer greater than or equal to 1 and smaller than or equal to 10;
C is LMWP, or polyarginine formed by 8 arginines;
D is selected from a small-molecule medicine;
D′ is selected from a monoclonal antibody and siRNAs having a length of oligonucleotide of 19-23 bp;
X is selected from —(CH₂)ⱼCONH(CH₂)ⱼ—, —(CH₂)ⱼ—S—S—(CH₂)ⱼ—, —(CH₂)ⱼNH—N═C(CH₂)ⱼ—, —(CH₂)ⱼCOO(CH₂)ⱼ— and —(CH₂)ⱼ—S—(CH₂)ⱼ—, wherein j is an integer of 0-5;
Z is selected from —(CH₂)ⱼCONH(CH₂)ⱼ—, —(CH₂)ⱼ—S—S—(CH₂)ⱼ—, —(CH₂)ⱼNH—N═C(CH₂)ⱼ—, —(CH₂)ⱼCOO(CH₂)ⱼ— and —(CH₂)ⱼ—S—(CH₂)ⱼ—, wherein j is an integer of 0-5; and
n is an integer of 3-14; k is an integer of 1-6; and p is an integer of 1-4.

16. The cell-penetrating-peptide multi-arm-PEG medicine conjugate having a general formula V according to claim 13, having a structure of:

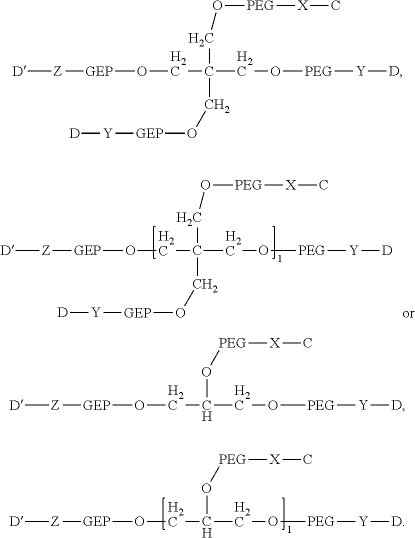

17-19. (canceled)
